(19) United States
(12) Patent Application Publication
(10) Pub. No.: US 2009/0053815 A1
(43) Pub. Date: Feb. 26, 2009

(54) Use of Holographic Sensors

(76) Inventors: Alexander James Marshall, Cambridge (GB); Simon Kew, Cambridge (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/088,882

(22) PCT Filed: Sep. 29, 2006
(86) PCT No.: PCT/GB06/03645

§ 371 Date: Aug. 4, 2008

(30) Foreign Application Priority Data
Oct. 3, 2005 (GB) 0520116.5

(51) Int. Cl. G01N 33/18; G01N 33/66; G01N 21/75

(52) U.S. Cl. 436/40; 436/164; 436/95; 422/55

(57) ABSTRACT

A method of measuring the amount of an analyte in a sample, comprises (a) passing the sample through a flow channel in or on a holographic sensor, wherein the analyte interacts with and is retained by the sensor and wherein the interaction of the analyte changes an optical property of the sensor, and (b) monitoring analyte interaction, wherein the distance along the flow channel at which analyte interaction occurs or the area of the channel over which analyte interaction occurs, indicates the amount of analyte in the sample. Further, a method of detecting an analyte in a sample, comprises (a) passing the sample through a flow channel in or on a holographic sensor, wherein the analyte interacts with the sensor, thereby changing an optical property of the sensor, and wherein the flow channel defines a symbol; and (b) detecting the analyte by observing the appearance, disappearance or change in appearance of the symbol.

### FIELD OF THE INVENTION

This invention relates to a method of measuring the amount of an analyte in a sample and to a device for use in such a method.

### BACKGROUND TO THE INVENTION

Holographic sensors are known, for example from WO95/26499 and WO03/087899, and may be used for the detection of a variety of analytes. An optical property of the sensor varies as the result of an interaction between the sensor and the analyte so that the presence of the analyte is indicated by the change in optical characteristics. To provide quantitative results, the degree of variation of the optical property is determined using a spectrometer.

### SUMMARY OF THE INVENTION

According to a first aspect of the present invention, a method of measuring the amount of an analyte in a sample comprises

(a) passing the sample through a flow channel in or on a holographic sensor, wherein the analyte interacts with and is retained by the sensor and wherein the interaction of the analyte changes an optical property of the sensor, and

(b) monitoring analyte interaction, wherein the distance along the flow channel at which analyte interaction occurs or the area of the channel over which analyte interaction occurs, indicates the amount of analyte in the sample.

According to a second aspect of the present invention, a device for use in a method according to a first aspect of the invention comprises a holographic sensor comprising sites at which an analyte can interact, wherein the sensor has an integrated flow channel through which a sample containing the analyte can pass.

According to a third aspect, a method of detecting an analyte in a sample, comprises

(a) passing the sample through a flow channel in or on a holographic sensor, wherein the analyte interacts with the sensor thereby changing an optical property of the sensor, and wherein the flow channel defines a symbol; and

(b) detecting the analyte by observing the appearance, disappearance or change in appearance of the symbol.

The method of the invention makes use of a flow channel to ensure that the sample is contacted with the sensor in a predetermined area along a defined path. The analyte in the sample interacts with, and becomes bound to, the support medium of the sensor as the sample passes through the flow channel hence decreasing the amount of analyte remaining in the fluid. The support medium responds to interaction with the analyte by changing an optical property of the sensor at a site relative to the site of analyte interaction, usually at the site of analyte interaction.

At some point along the flow channel, the sample will be substantially free of analyte as substantially all of the analyte initially present in the sample will have interacted with the sensor. Up to this point, the optical properties of the sensor will have changed due to interaction with the analyte. Beyond this point, there will be no interaction and hence no change in optical properties. This boundary or “response front” can be observed by monitoring the optical properties of the sensor along the flow channel. An optical filter can be used to modify the observed response of the sensor to make it easier to detect.

The area covered by the response and so the location of the response front is dependent on the amount of analyte in the sample (which is a result of the amount of fluid used and the initial concentration of analyte in the fluid). Hence, a quantitative indication of the amount of analyte in the sample is shown by the location of the response front along the flow channel. For any particular application, a holographic sensor is used which interacts with the analyte to be detected and the amount of analyte in the sample is selected to optimise the results by ensuring that there is a response front which is on an appropriate scale on the sensor.

The method of the present invention is advantageous as it allows for quantitative measurements to be made without a spectrometer. Spectrometers are expensive, inconvenient to transport and are, therefore, not suitable in a large number of situations. In contrast, the present invention provides a method of quantitative analysis that does not require any specialised detection equipment so it suitable for use in a wide range of applications, for example in a kit to be used in the home. Furthermore, the method gives rapid results and is easy to use.

### BRIEF DESCRIPTION OF THE DRAWINGS

FIGS. 1A to 1E relate to Example 1. FIGS. 1A to 1D show the detection of water in hexane at A) 0 ppm, B) 10 ppm, C) 20 ppm, D) 30 ppm respectively. FIG. 1E is a photograph of micro-machined flow channel used to define cross-shaped region where response is spatially targeted.

FIGS. 2A to 2F relate to Example 2. FIGS. 2A, B and C show the quantification of water droplets in kerosene using distance traveled along the flow channel to determine concentration. Water added to kerosene at the following levels: A: 0 ppm, B: 30 ppm, C: 120 ppm in Figures A, B, and C respectively. FIGS. 2D to 2F are diagrams of the system used.

FIGS. 3A to 3C relate to Example 3. FIG. 3A is a photograph of the response of a holographic film exposed to 100 ppm water in kerosene using a 1 mm annular flow head to display an O symbol. FIGS. 3B and 3C are diagrams of the system used.

### DETAILED DESCRIPTION OF THE INVENTION

The present invention is concerned with using a holographic sensor to indicate the presence of an analyte in a sample where the sensor is integrated with a flow channel to allow the amount of analyte to be detected.

A holographic sensor of the type used in the invention generally comprises a support medium and, disposed throughout the volume of the medium, a hologram. The support medium interacts with an analyte resulting in a variation of a physical property of the medium. This variation induces a change in an optical characteristic of the holographic sensor, such as its polarisability, reflectance, refractance or absorbance. The change causes a variation in an optical property, for example a colour or intensity change, which may be observed at a site which is relative to the site of analyte interaction. Usually the change in optical properties is at and around the site of analyte interaction.

The support matrix can be designed so that it binds with more than one type of analyte where the interaction with each type of analyte causes a characteristic change in an optical property of the sensor. Hence, the optical response could be used to identify, as well as quantify, the analyte in the sample.

There are a number of basic ways to change a physical property, and thus vary an optical characteristic. The physical property that varies is preferably the volume of the support medium and, in turn, the spacing of the holographic fringes of the holographic element. This variation may be achieved by incorporating specific groups into the support matrix, where these groups undergo a change in, for example, conformation, charge or the degree of cross-linking upon interaction with the analyte, and cause an expansion or contraction of the support medium. Such a group is preferably the specific binding conjugate of an analyte species.

In the invention the analyte is retained by the sensor, at least for the duration that the analyte is passing over the sample. This may be achieved by a chemical or physical interaction between the support and the analyte. Where the interaction is a chemical reaction, the analyte reacts with a specific group on the matrix and becomes bound to it. Alternatively, the interaction may be physical whereby the analyte become absorbed or adsorbed into or onto the sensor.

This retention of the analyte on the sensor may be reversed after the amount of analyte in a sample has been determined, by using a particular reagent or by subjecting the sensor to certain physical conditions. In this way, the sensor can be reused if desired, after it has been treated to remove the analyte.

A holographic sensor may be used for detection of a variety of analytes, simply by modifying the composition of the support medium. In the invention, the analyte may be, for example, water, glucose, lactate, a metal ion, an enzyme, an antibody, an alcohol or an aldehyde. Alternatively, the analyte may be RNA or DNA. The sample comprising the analyte may be aqueous or may comprise one or more organic solvents. Alternatively, the sample may be gaseous. The invention can be used to determine the, amount of water in a sample of kerosene, or the amount of glucose in a physiological fluid such as blood.

The medium preferably comprises a polymer matrix, the composition of which must be optimised to obtain a high quality film, i.e. a film having a uniform matrix in which holographic fringes can be formed. It is preferred that the medium is obtained by the (co)polymerisation of monomers including acrylamide-based monomers.

The polymer matrix is preferably substantially homogeneous. This means that the polymer matrix has the same or a similar level of cross-linking throughout and hence each area of the polymer exhibits substantially the same properties.

Other examples of holographic support media are gelatin, K-carageenan, agar, agarose, polyvinyl alcohol (PVA), sol-gels (as broadly classified), hydro-gels (as broadly classified), and acrylates. Further materials are polysaccharides, proteins and proteinaceous materials, oligonucleotides, RNA, DNA, cellulose, cellulose acetate, polyamides, polyimides and polyacrylamides. Gelatin is a standard matrix material for supporting photosensitive species, such as silver halide grains. Gelatin can also be photo-cross-linked by chromium III ions, between carboxyl groups on gel strands.

The change in optical properties caused by interaction of the sensor with an analyte (known as the response) should be an obvious and non-ambiguous change in the colour or image of the hologram, preferably in the visible region of the electromagnetic spectrum. This gives an accurate and reliable readout that can be observed by the naked eye. To help ensure that this is achieved, the sensor preferably has a optical filter thereon. The optical filter should cover some or all of the surface (or surfaces) of the sensor which are observed to monitor analyte interaction. The filter can be a lowpass filter (which allows radiation below a certain wavelength to pass through it), a highpass filter (which allows radiation above a certain wavelength to pass through it), or a bandpass filter (which allows radiation having a wavelength within a certain band, or certain bands in the case of a multi-bandpass filter, to pass through it). Hence, the use of such filters controls the frequency of the light that reaches the sensor. The hologram in the sensor acts like a bandpass reflector so the reflection wavelength of the hologram must be in the region of the filtered light to be transmitted back from the sensor to the observer or detector.

Filters are selected to provide a cut-off point for light of a high or low wavelength or both so can ensure that any response is in a particular range, for example, the visible range. They can be used to distinguish between different responses (for example to different analytes or analyte concentrations) which occur at different wavelength. They can also be used to prevent an ambiguous response if the sensor is used in non-optimal light conditions (for example, with monochromatic light). Optical filters can be specifically engineered to optimise the observed response to a specific analyte.

The hologram in the sensor of the invention can be generated by the diffraction of light. The hologram may only be visible under magnification, or may be viewable under white light, UV light or infra-red radiation or under specific temperature, magnetism or pressure conditions. The holographic image is preferably of an object or gives a 2- or 3-dimensional effect. The sensor may further comprise means for producing an interference effect when illuminated with laser light, preferably wherein the means comprises a depolarising layer.

The sensor may be prepared according to the methods disclosed in WO95/26499, WO99/63408 and WO03/087789. The contents of these specifications are incorporated herein by reference.

The sensor of the invention comprises an integrated flow channel. A sensor according to the invention may comprise more than one flow channel and can have many flow channels. A flow channel is any defined path on, in or through the sensor along which a sample can pass.

The flow channel or channels may be defined by the physical features of the hologram itself. This can be achieved by creating a groove in the material of the sensor or by using a sensor which is porous or perforated. Where the sensor is porous or perforated, each flow channel is defined by a perforation or a pore or a series of connected pores and the flow channel may pass through the sensor from one side to another.

Alternatively, in a different embodiment, the flow channel may be defined by an external fluidic device which is in contact with the sensor such as a microfluidic chip, moulded plastic chamber or porous material. The porous material may be constructed of beads, flakes, or fibres containing, or in contact with, the holographic sensor.

In either embodiment, the flow channel can be a linear groove of any size but often has dimensions of between 0.1 and 50 mm, preferably a width of 1 to 20 mm, a height of 0.1 to 1 mm and a length of 10 to 50 mm and, for example, is 10×0.5×20 mm. Where the channel is microfluidic it has micrometer dimensions, for example a width of 1 to 20 μm, a height of 0.1 to 1 μm and a length of 10 to 50 μm.

In one embodiment, the flow channel has dimensions which allow the sample to pass through it by capillary action. Suitable dimensions are dependent on the type of sample being tested but are typically in the order of 0.1 to 5 mm, for example about 0.5 mm. In this case, the sensor will be arranged so that, in use, the inlet of the flow channel is dipped into the sample which will enter and flow up the flow channel by capillary action. This is colloquially known as a “dipstick” method and is very convenient to use as can be hand-held and does not need any specialised equipment to control the flow of the sample in the channel so can be used at home. Examples of useful applications of such “dipstick” devices include using the sensor to test the alcohol content of a drink or the level of an analyte such as glucose in a physiological fluid such as blood.

It is preferred that the flow channel is a specific shape that is indicative of a result that can be obtained. In particular, the flow channel should be shaped so as to define a symbol. The symbol can be any two-dimensional pattern such as a letter, a number or a positive or negative symbol such as a tick or a cross. Such a flow channel can be advantageously used in a method for detecting an analyte in a sample or can be used in a method of measuring the amount of analyte in a sample.

According to a third aspect of the invention, the method is used to detect an analyte in a sample. In this case, it is not essential that the analyte is retained on the sample, provided that the analyte interacts with the sensor to change an optical property of the sensor. When analyte is present in the sample, it will cause the optical characteristics of the sensor in the region of the flow channel to change. Hence, the presence of the analyte will be observed as the appearance, disappearance or change in appearance of the symbol. An example of this would be a pregnancy test where a positive result is observed as a tick.

In the context of the first embodiment of the invention, the symbol could be a string of alphanumeric characters, for example, 1 to 5 wherein the amount of analyte can be determined by the highest number at which analyte interaction can be observed.

This embodiment is extremely useful as it gives a user-friendly visual read-out.

The sample is passed through the flow channel to enable the analyte to interact with the sensor. The level of analyte interaction will depend on the flow characteristics of the sample through the channel as, if the sample is passed through a shallow or narrow channel slowly, the analyte in the sample will have a higher likelihood of interacting with any particular interaction site on the sensor than if the sample is passed quickly though a wide deep channel. Therefore, the flow characteristics of the sample can be controlled to calibrate or adjust the sensitivity of the method.

As mentioned above, the flow channel may be such that the sample passes along it due to capillary action. Alternatively, the sensor may be arranged so that the sample passed along the flow channel under the action of gravity. In one embodiment, the sample is forced along the flow channel using a pump.

The analyte interaction can be monitored as a function of the flow rate of the sample through the channel to provide a way of varying the sensitivity of the sensor. In this embodiment, the sensor is tested with samples having a known amount of analyte at different flow rates. In this way, the sensor can be calibrated based on flow rate so that the relative distance along the channel or area over the channel at which analyte interaction occurs, as a function of the flow rate of the sample, gives an indication of the amount of analyte in the sample.

The analyte interaction is monitored by observing the optical property of the sensor which changes as a result of interaction with the analyte. The may be done with the naked eye or may be done using a detecting device. The device may also be used to store, transmit or process the optical data.

The device is preferably selected from the group consisting of an optical reader, a mobile phone, a computer and a digital camera. It is envisages that any type of computer can be used, such as a laptop, a desktop, or a hand held device such as a personal digital assistant (PDA) which is a personal organizer device. The optical change may be quantified using image analysis software.

An article comprising a device according to the invention can be used in various fields and is particularly useful in the security and medial fields. Such an article may be a transaction card, banknote, passport, identification card, smart card, driving license, share certificate, bond, cheque, cheque card, tax banderole, gift voucher, postage stamp, rail or air ticket, telephone card, lottery card, event ticket, credit or debit card, business card, or an item used in consumer, brand or product protection for the purpose of distinguishing genuine products from counterfeit products or identifying stolen products.

Alternatively, the article may be an item of intelligent packaging. “Intelligent packaging” refers to a system that comprises part of, or an attachment to, a container, wrapper or enclosure, to monitor, indicate or test product information or quality or environmental conditions that will affect product quality, shelf life or safety and typical applications, such as indicators showing time-temperature, freshness, moisture, alcohol, gas, physical damage and the like.

The invention can be used with an article which is an industrial or handicraft item comprising a decorative element, selected from items of jewellery, items of clothing (including footwear), fabric, furniture, toys, gifts, household items (including crockery and glassware), architecture (including glass, tile, paint, metals, bricks, ceramics, wood, plastics and other internal and external installations), art (including pictures, sculpture, pottery and light installations), stationery (including greetings cards, letterheads and promotional material) and sporting goods, or an article which is a product or device for use in agricultural studies, environmental studies, human or veterinary prognostics, theranostics, diagnostics, therapy, chemical analysis or petrochemical analysis, especially which is a test strip, chip, cartridge, swab, tube, pipette, contact lens, sub-conjuctival implant, sub-dermal implant, breathalyser, catheter or a fluid sampling or analysis device. The device of the invention can be included on a transferable holographic film. The film is preferably present on a hot stamping tape. The security of an article can be enhanced by transferring onto the article the device from the film.

The invention also relates to a product comprising a device of the invention which is capable of generating data from said device and to a system which uses data generated by such a product for data storage, control, transmission, reporting and/or modelling.

The following Examples illustrate the invention.

### Preparation of Holographic Film For Sensors in Examples 1 to 3

Holographic film was exposed to a collimated beam of HeNe irradiation at 632 nm. The exposed plates were developed for 2 min at 20° C. in the following developer solution: sodium hydroxide (20 g), anhydrous Na₂CO₃ (60 g), 4-(methylamino)phenol sulfate (Metol) (4 g) and ascorbic acid (30 g) dissolved in a total volume of 1 L of deionized water.

The developed silver-loaded plate was briefly rinsed in deionized water and immersed in a bleaching solution comprising sodium hydrogen sulfate (3 g), EDTA iron (III), sodium salt dihydrate (30 g) and potassium bromide (60 g) made up to 1 L in deionized water.

### EXAMPLE 1

This Example illustrates how dissolved water is detected in hexane. Water content determination in organic solvents is of importance in a wide range of industrial contexts where dryness is important.

200 mL of hexane, dried over molecular sieves, was allowed to equilibrate with ambient humidity, resulting in the gradual increase of its water content as determined by Karl-Fisher titration.

At various stages during this increase, at 10, 20 and 30 ppm, 5 mL of the hexane sample was drawn through a sensor device composed of a machined flow channel which encapsulates a holographic film such that it makes up one side of a flow channel 5 mm wide and 75 μm deep and configured such that a cross is formed.

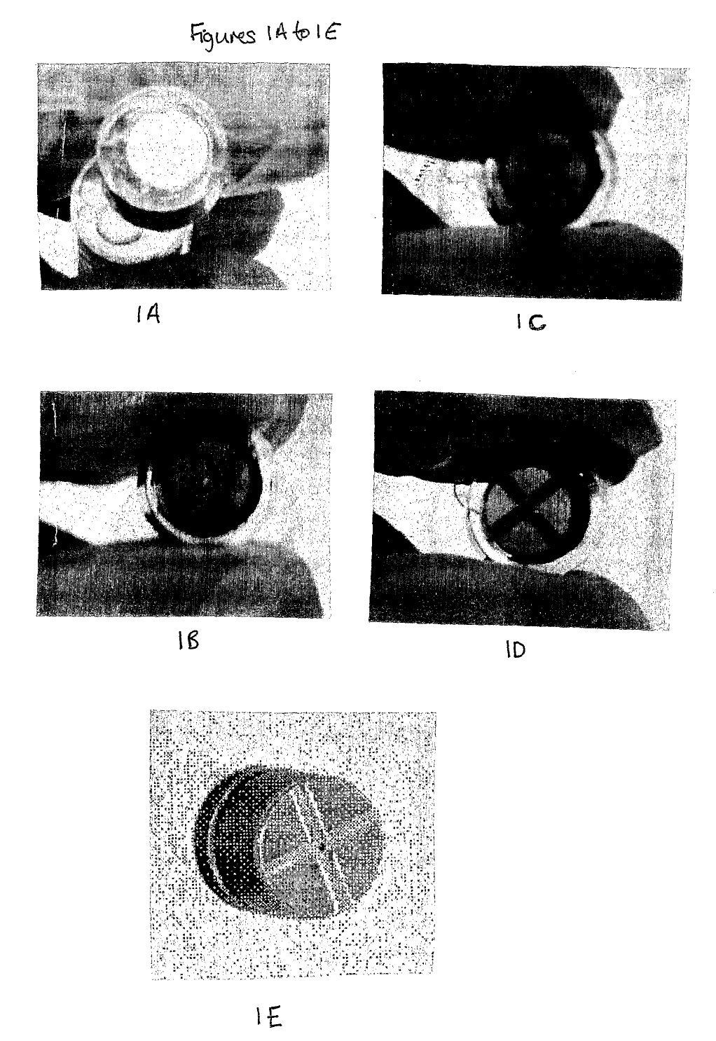

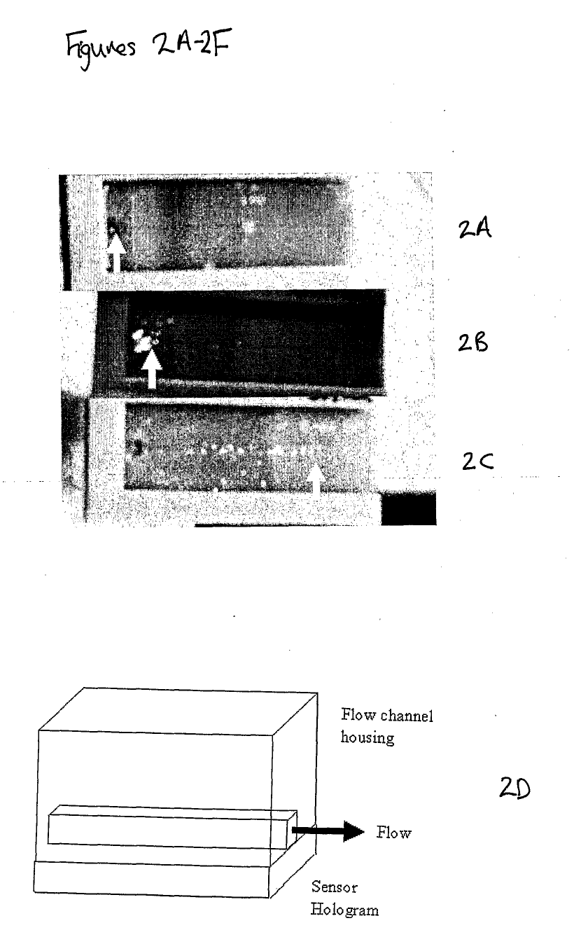

FIG. 1 shows the response of the sensor assembly to increasing levels of water in hexane, with a strong cross observed at 30 ppm. In this case, the response is a cross shape, but this approach enables any alphanumeric symbol be designed into the system

### EXAMPLE 2

In this example, bulk water droplets are detected in kerosene. This analysis is of significant importance in the petrochemical industry where entrained water in the fuel supply is a significant issue.

200 mL of kerosene was allowed to equilibrate with ambient humidity. To this was added various amounts of water and the mixture was vigorously shaken to generate a turbid emulsion containing water droplets and its water content was determined by Karl-Fisher titration.

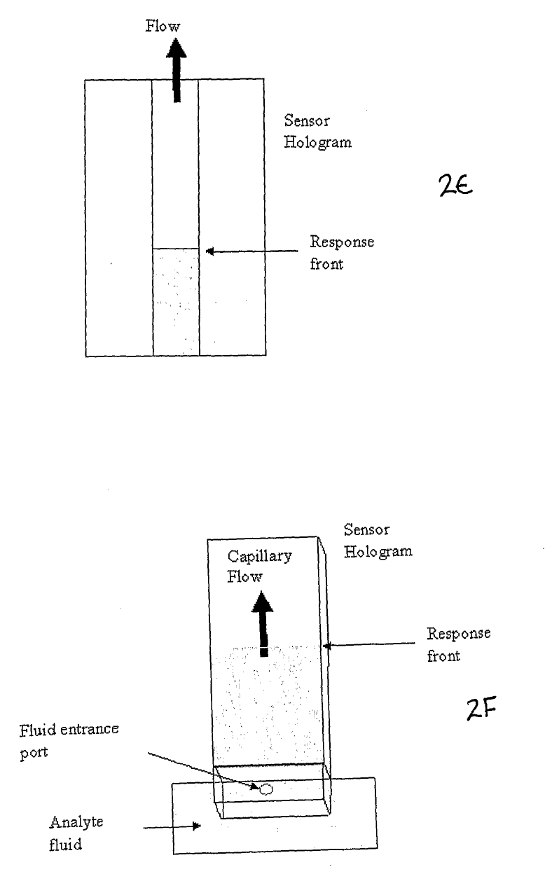

At various levels of water in the emulsion, at 0, 30 and 120 ppm in this example and shown in FIG. 2, 20 mL of the sample was flowed through a device composed of a hemispherical flow channel, with the flat side composed of the holographic sensor. The radius of the flow channel was 0.4-0.6 mm and the length 50 mm.

The flow channel was configured such that the distance along a flow channel at which a response is observed was used to give a semi-quantitative indication of the amount of an analyte that is present in the sample. This detection method is based on the idea that the analyte is sequestered by the detector and hence higher concentrations of analyte will travel further along the channel.

### EXAMPLE 3

In this example, emulsified water is detected in kerosene. This example demonstrates that alphanumeric characters can be displayed using a flow channel to direct flow into a sensor plate held normal to the direction of flow.

200 mL of kerosene was allowed to equilibrate with ambient humidity. To this was added various amounts of water and the mixture was shear-mixed to generate a turbid emulsion containing water droplets and its water content was determined by Karl-Fisher titration.

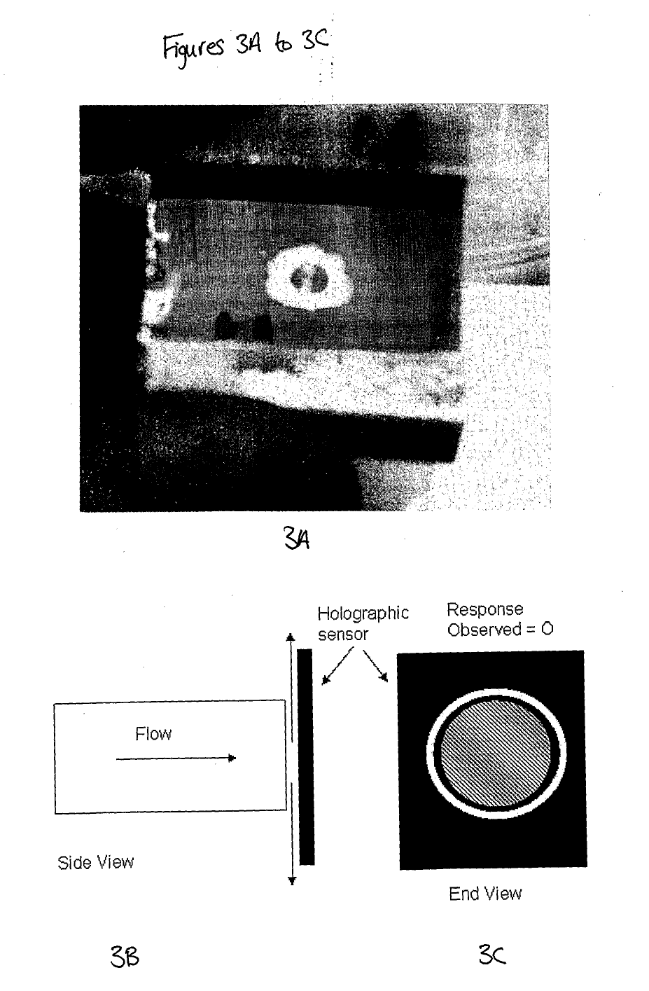

A circular annulus was placed in close proximity to the holographic plate such that a 0.1 mm gap was present between the circular annulus and the plate, normal to the direction of flow (see FIG. 3). A 100 ppm emulsion was flowed through the flow channel, resulting in the observation of an O shaped response on the holographic sensor film.

This invention also encompasses the idea that a profiled annulus can be used to spatially define the area where a response is to be observed. In this example, a circular annulus is used to display an O as the analyte is concentrated is a particular spatial region by the geometric configuration of the flow channel with respect to the holographic sensor. The shape of the annulus can be used to display a range of symbols.

Examples A to E below describe possible flow channels.

### EXAMPLE A

The device comprises a sensor hologram placed in intimate physical contact with a square section flow channel defined on three sides by a moulded plastic housing and defined on the fourth side by the sensor surface. The flow channel is a linear grove which is 0.5×10×20 mm. The distance travelled by the analyte “response front” enables quantification of analyte species.

### EXAMPLE B

The device comprises a sensor hologram placed in intimate physical contact with a microfluidic chip. The flow channels are defined by both the microfluidic chip and the surface of the sensor hologram. The microfluidic chip is composed of glass and contains parallel flow channels. The fluidic channels have micrometer dimensions with a width of 100 μm, a height of 50 μm and a length of 50 μm. The distance travelled by the analyte “response front” enables quantification of analyte species.

### EXAMPLE C

In this example, the fluidic channel is filled by capillary action. The flow channel is defined by a moulded plastic chamber of volume approximately 1 cm³. The chamber is attached to the sensor surface and is dipped into the fluid sample. The fluid enters the chamber via capillary action through an opening of approximately 5 mm in diameter in the chamber. The distance travelled by the analyte “response front” enables quantification of analyte species.

### EXAMPLE D

The flow channel is defined by perforation of the sensor hologram itself such that flow occurs through the sensor. In this example, the perforations are spherical holes 50 μm in diameter drilled through a holographic sensor film. The sample comprising the analyte is passed through the film of holographic sensor material which functions as a filter. The area covered by the analyte “response front” enables quantification of analyte species.

### EXAMPLE E

In this example, the holographic sensor is contained within or in contact with a porous material such that the flow channel is defined by a series of interconnected pores. In one such embodiment, the sensor hologram is coated with a fibrous material of average pore size around 10 μm, which acts to define a porous flow channel in contact with the sensor surface. In this embodiment, the fibrous material acts to deliver the analyte fluid to the sensor surface via capillary action.

1. A method of measuring the amount of an analyte in a sample, comprising
(a) passing the sample through a flow channel in or on a holographic sensor, wherein the analyte interacts with and is retained by the sensor and wherein the interaction of the analyte changes an optical property of the sensor, and
(b) monitoring analyte interaction, wherein the distance along the flow channel at which analyte interaction occurs or the area of the channel over which analyte interaction occurs, indicates the amount of analyte in the sample.

2. A method of detecting an analyte in a sample, comprising
(a) passing the sample through a flow channel in or on a holographic sensor, wherein the analyte interacts with the sensor, thereby changing an optical property of the sensor, and wherein the flow channel defines a symbol; and
(b) detecting the analyte by observing the appearance, disappearance or change in appearance of the symbol.

3. The method according to claim 2, wherein the symbol is an alpha-numeric character, a tick or a cross.

4. The method according to claim 1, wherein the analyte interaction is monitored as a function of the flow rate of the sample through the channel.

5. The method according to claim 1, wherein the analyte is a chemical, biochemical or biological species.

6. The method according to claim 1, wherein the analyte is water and the sample comprises an organic solvent or fuel.

7. The method according to claim 1, wherein the analyte is glucose and the sample comprises a physiological fluid.

8. The method according to claim 1, wherein the sensor can bind with more than one type of analyte and the interaction with each type of analyte causes a characteristic change in an optical property of the sensor.

9. The method according to claim 1, wherein the flow channel is a groove, perforation or pore in the holographic sensor.

10. The method according to claim 1, wherein the flow channel is a chamber on the holographic sensor.

11. The method according to claim 1, wherein, in step a), the sensor is dipped into the sample and the sample passes through the flow channel by capillary action.

12-21. (canceled)

22. A device suitable for use in a method selected from
i) a method of measuring the amount of an analyte in a sample, comprising
(a) passing the sample through a flow channel in or on a holographic sensor, wherein the analyte interacts with and is retained by the sensor and wherein the interaction of the analyte changes an optical property of the sensor, and
(b) monitoring analyte interaction, wherein the distance along the flow channel at which analyte interaction occurs or the area of the channel over which analyte interaction occurs, indicates the amount of analyte in the sample; and
ii) a method of detecting an analyte in a sample, comprising
(a) passing the sample through a flow channel in or on a holographic sensor, wherein the analyte interacts with the sensor, thereby changing an optical property of the sensor, and wherein the flow channel defines a symbol; and
(b) detecting the analyte by observing the appearance, disappearance or change in appearance of the symbol;
wherein said device comprises a holographic sensor comprising sites at which an analyte can interact, wherein the sensor has an integrated flow channel through which a sample containing the analyte can pass.

23. The device according to claim 22, wherein the flow channel defines a symbol.

24-34. (canceled)

35. The method according to claim 2, wherein the analyte interaction is monitored as a function of the flow rate of the sample through the channel.

36. The method according to claim 2, wherein the analyte is a chemical, biochemical or biological species.

37. The method according to claim 2, wherein the analyte is water and the sample comprises an organic solvent or fuel.

38. The method according to claim 2, wherein the analyte is glucose and the sample comprises a physiological fluid.

39. The method according to claim 2, wherein the sensor can bind with more than one type of analyte and the interaction with each type of analyte causes a characteristic change in an optical property of the sensor.

40. The method according to claim 2, wherein the flow channel is a groove, perforation or pore in the holographic sensor.

41. The method according to claim 2, wherein the flow channel is a chamber on the holographic sensor.

42. The method according to claim 2, wherein, in step a), the sensor is dipped into the sample and the sample passes through the flow channel by capillary action.
